(19) United States
(12) Patent Application Publication
(10) Pub. No.: US 2004/0033256 A1
(43) Pub. Date: Feb. 19, 2004

(54) Liposome-encapsulated insulin formulations

(76) Inventor: Rimona Margalit, Givatayim (IL)

Correspondence Address: BOZICEVIC, FIELD & FRANCIS LLP, 200 MIDDLEFIELD RD, SUITE 200, MENLO PARK, CA, 94025, US

(21) Appl. No.: 10/641,953

(22) Filed: Aug. 15, 2003

Related U.S. Application Data
(62) Division of application No. 09/975,752, filed on Oct. 9, 2001, now abandoned.

(51) Int. Cl. A61K 38/28; A61K 9/127

(52) U.S. Cl. 424/450; 514/3

(57) ABSTRACT

The present invention provides formulations of insulin or insulin analogs encapsulated in a liposome, and methods of producing such formulations. The invention further provides methods of treating hyperglycemia and related disorders by administering a formulation of the invention.

### FIELD OF THE INVENTION

This invention relates generally to liposomal insulin formulations, and methods of treating hyperglycemia and related conditions using the formulations.

### BACKGROUND OF THE INVENTION

There are several metabolic diseases of human and animal glucose metabolism, eg., hyperglycemia, insulin dependent diabetes mellitus, impaired glucose tolerance, hyperinsulinemia, and insulin insensitivity, such as in non-insulin dependent diabetes mellitus (NIDDM). Hyperglycemia is a condition where the blood glucose level is above the normal level in the fasting state, following ingestion of a meal or during a glucose tolerance test. It can occur in NIDDM as well as in obesity. Hyperglycemia can occur without a diagnosis of NIDDM. This condition is called impaired glucose tolerance or pre-diabetes. Impaired glucose tolerance occurs when the rate of metabolic clearance of glucose from the blood is less than that commonly occurring in the general population after a standard dose of glucose has been orally or parenterally administered. It can occur in NIDDM as well as obesity, pre-diabetes and gestational diabetes. Hyperinsulinemia is defined as having a blood insulin level that is above normal level in fasting state or following ingestion of a meal. It can be associated with or causative of hypertension or atherosclerosis. Insulin insensitivity, or insulin resistance occurs when the insulin-dependent glucose clearance rate is less than that commonly occurring in the general population during diagnostic procedures.

Diabetes mellitus is a disease affecting approximately 150 million persons worldwide. Of the 7.5 million diagnosed diabetics in the United States, approximately one-third are treated using insulin replacement therapy. Those patients receiving insulin typically self-administer one or more doses of the drug per day by subcutaneous injection.

Diabetes mellitus is classified into two types, type I diabetes or insulin-dependent diabetes mellitus (IDDM) and type II diabetes or non-insulin-dependent diabetes mellitus (NIDDM). Type I diabetes, in which the pancreas has stopped producing insulin, affects 10% of all diabetics, often begins in childhood and is known as juvenile onset diabetes. In the more prevalent type II diabetes, affecting 90% of all diabetics, the pancreas can produce insulin, but insulin secretion in response to meals is diminished, and the diabetic's tissues are not as responsive to insulin as tissues from a non-diabetic. Type II diabetes is also known as adult onset diabetes.

Insulin is a polypeptide with a nominal molecular weight of about 6,000 Daltons. Insulin has traditionally been produced by processing pig and cow pancreas to allow isolation of the natural product. More recently, recombinant technology has made it possible to produce human insulin in vitro. It is the currently common practice in the United States to institute the use of recombinant human insulin in all of those patients beginning insulin therapy.

It is known that most proteins are rapidly degraded in the acidic environment of the gastrointestinal (GI) tract. Since insulin is a protein that is readily degraded in the GI tract, those in need of the administration of insulin administer the drug by subcutaneous injection. No satisfactory method of orally administering insulin has been developed. The lack of such an oral delivery formulation for insulin creates a problem in that the administration of drugs by injection can be both psychologically and physically painful.

Many investigators have studied alternate routes for administering insulin, such as oral, rectal, transdermal, and nasal routes. So far, these types of administration have not been effective due to poor and variable insulin absorption, lack of significant decrease in serum glucose levels, or irritation at the site of delivery. In contrast, pulmonary delivery of fine particle aerosols delivered with appropriate control of the patient's breathing results in effective and reproducible absorption of insulin and reduction of glucose level similar to the response obtained by subcutaneous injection of short-acting (“regular”) insulin. However, currently the plasma profiles following pulmonary administration of these insulin formulations indicates that these methods do not achieve sustained levels of insulin that could replace injections of long acting insulins.

Thus, there is a need in the art for a long-acting insulin formulation, and in particular for a long-acting insulin formulation that may be aerosolized for pulmonary administration.

Literature

Yoshida et al. (1979) J. Pharm. Sci. 68:670-671; Moses et al. (1983) Diabetes 32:1040-1047; Salzman (1985) New England Journal of Medicine 312:1078-1084; Farr et al. (2000) Diabetes Technology and Therapeutics 2:185; Laube (1991) Journal of Aerosol Medicine 4:286; Colthorpe (1992) Pharmaceutical Research 9:764-768; Wigley (1971) Diabetes 20:552-556; Kohler (1990) Lung 168 Suppl.:677-684; Brunner et al. (2000) Diabetologia 44:305-308; Heinemann et al. (2000) Diabetes Care 23 :1343-1347; Gonda (2000) J. Pharm. Sci. 89:940-945; and Patton et al. (1999) Adv. Drug. Deliv. Rev. 35:235-247; and U.S. Pat. Nos. 5,672,581; 5,888,477; 5,514,646; 5,504,188; 5,545,618; 6,034,054; 5,970,973; and 5,888,477.

### SUMMARY OF THE INVENTION

The present invention provides formulations of insulin or insulin analogs encapsulated in a liposome, and methods of producing such formulations. The invention farther provides methods of treating hyperglycemia and related disorders by administering a formulation of the invention.

One embodiment of the invention is a method for formulating insulin for use as a pharmaceutical, where an insulin solution having a basic pH is produced, the solution is encapsulated in liposomes at a basic pH, and subsequent to encapsulation, the liposomes are neutralized to a more neutral pH, e.g., a pH of 7.2 to 7.6. The encapsulation at the basic pH provides a high efficiency of encapsulation of the insulin or insulin analog, and the neutralization following encapsulation provides a formulation that is of a desired pH for administration to a patient.

In other embodiments, the invention provides an acidic to neutral approach to generating an insulin-containing liposome formulation, in which dry insulin is dissolved under acidic conditions to a pH in the range of about 2-3, the preparation is then titrated by NaOH, typically under conditions that will minimize dilution, to about pH 7.2-7.4. The preparation is then generally buffered.

Another embodiment of the invention is an insulin formulation produced using the methods of the invention. In a preferred embodiment, the formulation provides a high insulin dose, e.g., from about 500-1500 insulin units/ml. The formulation of this preferred embodiment will generally be composed of 20 to 80 mg/ml insulin in the formulation. The formulation may be produced as a solution (e.g., an aqueous solution), as a dry powder, as a colloidal suspension, and the like. In a particular embodiment, the formulation is aerosolized and particles are produced which preferably have an aerodynamic diameter in a range from 1 to 5 microns.

Another embodiment of the invention is a method for treating hyperglycemia and related disorders by administering an insulin formulation of the invention to a patient. Another embodiment of the invention is a method for treating diabetes mellitus in a patient by administering an insulin formulation of the invention to a patient. In some embodiments, the formulation is administered to the patient via an aerosol, e.g., via an aqueous aerosol.

An advantage of the present invention is that the encapsulation efficiencies are higher than encapsulation at a more neutral pH.

Another advantage of the present invention is that the formulations are sustained release, long-acting formulations.

A feature of the formulations of the present invention is that they can be designed to form particles which when inhaled localize deep within the lungs.

Another feature of the formulations of the present invention is that the insulin released from the liposomes is absorbed into the blood stream.

These and other objects, advantages, and features of the invention will become apparent to those persons skilled in the art upon reading the details of the subject invention as more fully described below.

Features of the Invention

The invention features methods for preparing an insulin formulation. The methods generally involve the steps of: (a) preparing a solution comprising insulin, wherein the solution has a non-neutral pH; (b) encapsulating the solution in liposomes at a non-neutral pH; and (c) neutralizing the liposomes of step (b) to pH of 7.2 to 7.6. In some embodiments, the solution has a pH between about 2.3 and 3.0 before it is neutralized, and the encapsulation takes place in an acidic environment. In other embodiments, the solution has a pH between about 7.8 and 9.5 before it is neutralized, and the encapsulation takes place in a basic environment. In many embodiments, the solution of step (a) includes 20 to 80 mg/ml insulin. In many embodiments, the liposomes are between about 0.2 and 3 microns in diameter.

In some embodiments, the liposomes comprise a lipid selected from the group consisting of: phosphatidylcholine, phosphatidyl ethanolamine, cholesterol, phosophatidyl serine, phosphatidyl glycerol and phosphatidyl inositol. In some embodiments, the liposomes are unilamellar. In other embodiments, the liposomes are multilamellar.

The invention further features a composition comprising monomeric insulin encapsulated in liposomes, wherein the monomeric insulin has a molecular weight of about 6,000 Da. In some embodiments, the insulin is an insulin analog. In some of these embodiments, the insulin is selected from the group consisting of a superactive insulin analog, a monomeric insulin analog, and a hepatospecific insulin analog. In other embodiments, the encapsulated insulin is insulin lispro. In some embodiments, the solution further comprises a second therapeutic agent.

The invention further features a method for reducing a blood glucose level in a patient. The method generally involves administering a formulation comprising monomeric insulin encapsulated in liposomes to the patient. In some embodiments, the monomeric insulin has a molecular weight of about 6,000 Da. In some embodiments, the administering is carried out by creating an aerosol of the formulation; and inhaling the aerosol. In other embodiments, the administering is carried out by creating an injectable solution of the formulation; and injecting the solution.

The invention further features a method for treating a condition related to hyperglycemia in an individual. The method generally involves administering a formulation comprising monomeric insulin encapsulated in liposomes to the individual.

The invention further features a method of delivering insulin to a lung of an individual. The method generally involves preparing an insulin formulation by a method of the invention; aerosolizing the insulin formulation to provide aerosol particles having an aerodynamic diameter of about 1 to about 5 microns; and inhaling the aerosol into the lung of the individual.

### BRIEF DESCRIPTION OF THE DRAWINGS

FIG. 1 is a graph depicting kinetics of insulin efflux from liposomes. “Total” indicates the complete liposome preparation, at a liposome concentration of 100 mg lipid/ml; “Total/20” indicates a liposome concentration of 5 mg lipid/ml; “Pellet” indicates a sample from the complete preparation at a liposome concentration of 100 mg/ml after removal of unencapsulated insulin by centrifugation. The points are experimental and the solid curves are the theoretical expectations according to equation (2) in the text.

FIG. 2 is a graph depicting the rate constant for efflux of encapsulated insulin, k₂, as function of liposome concentration expressed in mg lipid/ml. The points are experimental, the error bars representing the standard deviations. The solid line is non theoretical, drawn to emphasize the trend of the data.

### DETAILED DESCRIPTION OF THE INVENTION

The present invention provides formulations of insulin or insulin analogs encapsulated in a liposome, and methods of producing such formulations. The invention further provides methods of treating hyperglycemia and related disorders by administering a formulation of the invention.

In particular, the present invention provides sustained release, long-acting formulations comprising insulin formulated in a liposome, and methods of producing such liposomal formulations. Liposome-encapsulated insulin formulations of the invention are characterized by a high concentration of insulin. Thus, the amount of formulation that need be administered is reduced. This is advantageous, as the number of required dosing events is reduced.

### Definitions

As used herein, the term “insulin” refers to natural extracted human insulin; recombinantly produced human insulin; insulin extracted from bovine and/or porcine sources; recombinantly produced porcine and bovine insulin; insulin analogs; derivatized insulin; insulin derivatives produced synthetically or semi-synthetically; and combinations of any of the foregoing. The term is intended to encompass the polypeptide normally used in the treatment of diabetics in a substantially purified form, and also encompasses the use of the term in its commercially available pharmaceutical form which includes additional excipients. In some embodiments, the insulin used to generate the insulin-containing liposomes described herein is recombinantly produced human insulin. The term “insulin” is also intended to encompass insulin analogs which include any form of insulin wherein one or more of the amino acids within the polypeptide chain has been replaced with an alternative amino acid and/or wherein one or more of the amino acids has been deleted or wherein one or more additional amino acids has been added to the polypeptide chain. In general, the “insulin analogs” of the present invention include monomeric insulin analogs, such as insulin lispro, “super insulin analogs”, wherein the ability of the insulin analog to affect serum glucose levels is substantially enhanced as compared with conventional insulin as well as hepatoselective insulin analogs which are more active in the liver than in adipose tissue. Insulin derivatives include, but are not limited to, acylated derivatives.

The term “dosing event” shall be interpreted to mean the administration of insulin and/or an insulin analog to a patient in need thereof by the intrapulmonary route of administration which event may encompass one or more releases of insulin formulation from an insulin dispensing device over a period of time of 15 minutes or less, preferably 10 minutes or less, and more preferably 5 minutes or less, during which period one or multiple inhalations are made by the patient and one or multiple doses of insulin are released and inhaled. A dosing event shall involve the administration of insulin to the patient, and/or absorption into the patient of insulin, in an amount of about 1 unit to about 50 units in a single dosing event, which may involve the release of from about 1 to about 500 units of insulin from a delivery device.

The term “measuring” describes an event whereby either or both the inspiratory flow rate and inspiratory volume of the patient is measured in order to determine an optimal point in the inspiratory cycle at which to release aerosolized insulin formulation. It is also preferable to continue measuring inspiratory flow during and after any drug delivery and to record inspiratory flow rate and volume before, during and after the release of drug. Such reading makes it possible to determine if the insulin formulation was properly delivered to the patient. A microprocessor or other device can calculate volume based on a measured flow rate. When either flow rate or volume becomes known in any manner it can be said to have been determined. Thus, it may be measured electrically, mechanically, or by coaching a patient to breathe in a particular manner.

The term “monitoring” event shall mean measuring lung functions such as inspiratory flow, inspiratory flow rate, and/or inspiratory volume so that a patient's lung function as defined herein, can be evaluated before and/or after drug delivery thereby making it possible to evaluate the effect, if any, of insulin delivery on the patient's lung function.

The terms “formulation” and “liquid formulation” and the like are used interchangeably herein to describe any liposome-encapsulated insulin, derivatized insulin, or insulin analog, alone or in combination with another drug for treating diabetes mellitus by itself or with a pharmaceutically acceptable carrier. Such formulations are preferably solutions, e.g., aqueous solutions, saline solutions and colloidal suspensions. Formulations can be solutions or suspensions of the liposomes of the invention in a low boiling point propellant.

The term “aerosol” means particles of a formulation wherein the particles have an aerodynamic diameter in the range of 0.1 to 10 μm, preferably 1 to 5 μm, and preferably the total volume of aerosolized formulation in each dose is from 5 μl to 10,000 μl. About 10 μl to about 50 μl of particles having an aerodynamic diameter of about 1 to 3 microns are present in a volume of inhaled air of about 50 ml to 2 liters, preferably 100 ml to 1,000 ml.

The terms “air”, “particle free air”, “aerosol free air,” and the like, are used interchangeably herein to describe a volume of air that is substantially free of other material and, in particular, free of particles intentionally added such as particles of formulation which create the aerosol. The term means that the air does not include particles of formulation which have been intentionally added but is not intended to imply that the normal surrounding air has been filtered or treated to remove all particles although filtering can take place. Air is the preferred gas to use with drug delivery, it being noted that other non-toxic gases, e.g., pure oxygen, propellants, and CO₂ can be used.

The terms “particles”, “aerosolized particles” and “aerosolized particles of formulation” are used interchangeably herein to refer to particles of formulation encapsulated in a liposome. The particles have a size that is sufficiently small such that when the particles are formed they remain suspended in the air for a sufficient amount of time such that the patient can inhale the particles into the patient's lungs. The particles have a size in the range of from about 0.1 μm to about 50 μm, generally from about 0.5 μm to about 10 μm. Particle diameter is an aerodynamic diameter.

The term “substantially dry” shall mean particles of an aerosol that contain less than 10% free water, ethanol or other liquid carrier based on total weight and preferably contains no detectable free liquid carrier.

As used herein, the term “hyperglycemia” refers to an above-normal level of glucose in the blood, where a normal level is in the range of from about 65 mg/dL to about 140 mg/dL. Generally, hyperglycemia refers to a blood glucose level in excess of about 140 mg/dL.

As used herein, the term “a condition associated with hyperglycemia” refers to a condition that is a result of hyperglycemia, a condition of which hyperglycemia is a symptom, and a condition that results from a condition associated with hyperglycemia. Such conditions include, but are not limited to, impaired glucose tolerance; insulin dependent diabetes mellitus (IDDM; Type I diabetes); non insulin dependent diabetes mellitus (NIDDM: Type II diabetes); insulin resistance; insulin insensitivity; disorders associated with diabetes, including but not limited to, diabetic nephropathy, diabetic neuropathy, diabetic retinopathy, cardiovascular disorders, cerebrovascular disorders, periodontal disease, and hypertension.

As used herein, the terms “treatment”, “treating”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse affect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

The terms “individual,” “host,” “subject,” and “patient,” used interchangeably herein, refer to a mammal, including, but not limited to, murines, rodents, lagomorphs, simians, humans, ungulates, felines, canines, mammalian farm animals, mammalian sport animals, and mammalian pets.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a formulation” includes mixtures of different formulations, reference to “an insulin analog” refers to one or mixtures of insulin analogs, and reference to “the method of treatment” includes reference to equivalent step and methods known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Liposomal Insulin Formulations and Methods of Producing Same

The present invention provides sustained release, long-acting formulations comprising insulin formulated in a liposome, and methods of producing such liposomal formulations. Liposome-encapsulated insulin formulations of the invention are characterized by a high concentration of insulin. Thus, the amount of formulation that need be administered is reduced. This is advantageous, as the number of required dosing events is reduced.

Neutral insulin solutions at the concentration range of U500-U1500 (3-10 mM) exhibit a high degree of reversible self-aggregation, predominantly hexamers and higher-order aggregates, i.e. six insulin molecules complexed together. The present invention provides a method for formulating insulin in liposomes under conditions favorable to the encapsulation of insulin monomers from any source of insulin. The resulting formulation can be used for any method of administration, including but not limited to intravenous, intramuscular, inhalation, oral, buccal, nasal, subcutaneous, etc. In particular, the invention provides the unexpected result that encapsulation of insulin into liposomes, initiated under acidic or basic conditions (where the insulin is much less aggregated than at neutral pH) reduces the effective size of the dominant encapsulated species from 36,000 Da (or more) towards 6000 Da, the size of insulin monomers. This affords better encapsulation of insulin than if initiated at neutral pH. Thus, the encapsulation conditions of the invention drive dissociation of the insulin aggregates towards encapsulation in the monomeric form.

In some embodiments, the liposome-encapsulated insulin is administered to a patient in an aerosol inhalation device. In some embodiments, insulin is encapsulated in the liposomes in combination with other pharmaceuticals. In some embodiments, the liposomes are administered in combination with insulin that is not encapsulated, with pharmaceuticals that are not encapsulated, or various combinations thereof.

A formulation of the invention is generally prepared by the process of combining a lipid composition with an insulin solution, suspension or mixture at a non-neutral pH which is basic, e.g., in a pH range of from about 7.8 to about 9.5, or, alternatively, which is acidic, e.g., in a pH in a range of 2-3; and mixing the lipid composition with the insulin solution in a manner so as to obtain liposomes which contain insulin.

The insulin component of the subject liposomal formulations consists of at least about 10%, at least about 20%, at least about 30%, at least about 40%, at least about 50%, at least about 60%, at least about 70%, at least about 75%, at least about 80%, at least about 85%, at least about 90%, at least about 95%, at least about 98%, or more, monomeric insulin, with the remaining insulin in higher molecular weight, e.g., aggregated, forms, such that the insulin component consists of about 2%, about 5%, about 10%, about 15%, about 20%, about 25%, about 30%, about 40%, about 50%, about 60%, about 70%, about 80%, about 90%, or more, of a higher molecular weight form. Thus, an aspect of the invention is a pharmaceutical formulation which contains a pharmaceutically acceptable carrier and liposomes comprised of a lipid and insulin.

Using the methods of the invention for liposomal encapsulation of insulin, the efficiency of encapsulation is at least about 50%, at least about 55%, at least about 60%, at least about 65%, at least about 70%, at least about 75%, at least about 80%, at least about 85%, at least about 90%, at least about 95%, or higher. By “encapsulation efficiency” is meant that at least about 50%, at least about 55%, at least about 60%, at least about 65%, at least about 70%, at least about 75%, at least about 80%, at least about 85%, at least about 90%, at least about 95%, or more, of the insulin is encapsulated in liposomes.

The concentration of insulin in the subject liposomal formulations is in the range of from about 250 to about 3000, from about 300 to about 2500, from about 400 to about 2000, or from about 500 to about 1500 Units/ml. In general, the concentration of insulin in the subject liposomal formulations is from about 25 to about 75, from about 30 to about 70, or from about 40 to about 60 mg/ml.

In some embodiments, the invention provides a “basic to neutral” method to generating an insulin-containing liposome formulation, in which dry insulin is dissolved in a buffer under basic conditions to a pH in the range of from about 7.8 to about 9.5, and to a concentration in the range of 20 to 80 mg/ml.

Multilamellar vesicles (MLV) are prepared according to techniques well known in the art. Briefly, in one embodiment, lipids are weighed and dissolved in a suitable organic solvent (such as chloroform or a chloroform-methanol mixture). The organic solvent is evaporated to complete dryness in a rotary evaporator, under low pressure, and at a temperature range of about 37-40° C. Following evaporation, the insulin solution (“swelling solution”) is added to the dry lipid film. The system is vigorously mixed, then incubated for about two hours in, for example, a shaker bath at a temperature range appropriate for the lipid composition. This basic MLV preparation is then titrated by HCl (or other suitable acid such as sulfuric acid or acetic acid), preferably under conditions that will minimize dilution, to about pH 7.2-7.4. The preparation can then be buffered, for example, by adding about a one tenth volume of ten-fold concentrated phosphate buffered saline (PBS) or other suitable buffer, of about pH 7.4. An example of this protocol is found in Example 7.

A wide variety of buffers are known to those skilled in the art, and it is well within the skill level of those skilled in the art to select appropriate buffering agent for a desired pH range. Non-limiting examples of materials suitable for biological (or biological-derived) matter for buffering at basic pH in the range of 7.8-9.5 include: GlyGly (glycylglycine) at concentrations of 2.64 mg/ml and up to 0.2 M. Glycine-NaOH or Tris-HCl, both from 10 mM up to 0.2 M. Non-limiting examples of materials suitable for biological (or biological-derived) matter for buffering at the neutral pH range of 7.0-7.6 include: Phosphate-buffer, buffer salts at 3-7 mM; and Tris-HCl and HEPES buffers from 10 mM-0.2 M.

In other embodiments, the invention provides an “acidic to neutral” approach to generating an insulin-containing liposome formulation, in which dry insulin is dissolved under acidic conditions to a pH in the range of about 2-3, and to a concentration of between 20-80 mg/ml. MLV are prepared as described above, except that the MLV preparation is acidic. This acidic MLV preparation is then titrated by NaOH, preferably under conditions that will minimize dilution, to about pH 7.2-7.4. The preparation is then preferably buffered, for example, by adding about a one tenth volume of ten-fold concentrated phosphate buffered saline (PBS), of pH 7.4.

In one embodiment, MLV generated as described above serve as the source material for acidic unilamellar vesicles (ULV). For example, MLV are prepared as described above and subjected to extrusion in a device such as, for example, that manufactured by Lipex Biomembranes, Inc. (Vancouver, British Columbia). Extrusion is performed through a series of membranes with progressively-smaller pore sizes, such as, for example, starting with pore sizes in the range of 0.8 to 1.0 μm (one to two extrusion cycles per pore size) and ending at the pore size range selected according to the desired liposome size (e.g., about seven cycles of extrusion at the final pore size). In some embodiments, the starting MLV material is neutralized before extrusion. In other embodiments, the MLV is subjected to extrusion while at an acidic or basic pH. In these latter embodiments, the acidic or basic ULV extrusion product is titrated to a neutral pH as described above.

The liposomes of the invention may be any appropriate form known to those skilled in the art used in the art, including multilamellar and unilamellar. See e.g., Liposomes, Ed. R.R.C. New, Oxford University Press, New York (1997), and Gregoriadis, Liposome Technology, volumes I-III, CRC Press, Boca Raton, Fla. (1984) which are incorporated herein by reference. The techniques used in the present invention can use almost any lipid composition that makes liposomes upon appropriate suspension of the lipids in a hydrophilic solution, e.g., water. Exemplary lipids that can be used to produce the liposomes of the formulations of the invention include (i) phosphatidylcholine alone or mixed with lipids such as: phosphatidyl ethanolamine, cholesterol, phosphatidyl serine, phosphatidyl glycerol and phosphatidyl inositol; and (ii) charged phospholipids such as phosphatidyl serine, phosphatidyl glycerol and phosphatidyl inositol, each alone or as mixtures.

In general, the size of the liposomes generated is preferably about 0.2 to 3 micrometer in diameter.

In general, any form of insulin may be utilized in the formulations of the instant invention, as long as the insulin is biologically active, i.e., the insulin is effective in reducing blood glucose levels in an individual who is responsive to insulin. In some embodiments, recombinant human insulin (“regular” insulin) or a recombinant human insulin analog is used. In a particular embodiment, the insulin analog is a monomeric form of insulin, e.g., human lispro. In some instances, other forms of insulin are used alone or in combination with recombination human insulin or each other. Insulin that is suitable for use herein includes, but is not limited to, regular insulin, semilente, NPH, lente, protamine zinc insulin (PZI), ultralente, insuline glargine, insulin aspart, acylated insulin, monomeric insulin, superactive insulin, hepatoselective insulin, and any other insulin analog or derivative, and mixtures of any of the foregoing. Insulin that is suitable for use herein includes, but is not limited to, the insulin forms disclosed in U.S. Pat. Nos. 4,992,417; 4,992,418; 5,474,978; 5,514,646; 5,504,188; 5,547,929; 5,650,486; 5,693,609; 5,700,662; 5,747,642; 5,922,675; 5,952,297; and 6,034,054; and published PCT applications WO 00/121,197; WO 09/010,645; and WO 90/12814. Insulin analogs include, but are not limited to, superactive insulin analogs, monomeric insulins, and hepatospecific insulin analogs.

Superactive insulin analogs have increased activity over natural human insulin. Accordingly, such insulin can be administered in substantially smaller amounts while obtaining substantially the same effect with respect to reducing serum glucose levels. Superactive insulin analogs include, e.g., 10-Aspartic Acid-B human insulin; des-pentapeptide (B26-B30)-,Asp^{b10}, Tyr^{B25}-α-carboxamide human insulin, (B26-B30)-,glu^{B10,} Tyr^{B25}-α-carboxamide human insulin, and further insulin analogs of the formula des(B26-B30)-, X^{B10}, Tyr^{B25}-α-carboxamide human insulin, in which X is a residue substituted at position 10 of the B chain. These insulin analogs have potencies anywhere from 11 to 20 times that of natural human insulin. All of the above-described insulin analogs involve amino acid substitutions along the A or B chains of natural human insulin, which increase the potency of the compound or change other properties of the compound. Monomeric insulin includes, but is not limited to, lispro.

Insulin derivatives include, but are not limited to, acylated insulin, glycosylated insulin, and the like. Examples of acylated insulin include those disclosed in U.S. Pat. No. 5,922,675, e.g., insulin derivatized with a C₆-C₂₁ fatty acid (e.g., myristic, pentadecylic, palmitic, heptadecylic, or stearic acid) at an α- or ε-amino acid of glycine, phenylalanine, or lysine.

Methods of Treating Hyperglycemia and Related Disorders

The invention further provides methods of treating hyperglycemia, and conditions related to hyperglycemia. The methods generally involve administering an effective amount of a liposomal insulin formulation of the invention to a subject.

A liposomal insulin formulation of the invention is administered to an individual in a therapeutically effective amount, e.g., an amount that is effective to treat hyperglycemia and/or a condition associated with hyperglycemia such that at least one measure of hyperglycemia or a condition associated with hyperglycemia is reduced by at least about 5%, at least about 10%, at least about 20%, at least about 30%, at least about 40%, at least about 50%, at least about 60%, at least about 70%, at least about 80%, at least about 90%, or more, compared with an untreated individual. For example, if the measure is blood glucose level, and the blood glucose level is 250 mg/ml, an effective amount of a subject formulation is effective to reduce blood glucose level to 240 mg/ml or less, preferably to reduce the blood glucose level such that it is within the normal range, e.g., from about 65 mg/dL to about 140 mg/dL. Thus, in some embodiments, the present invention provides methods for reducing a blood glucose level in an individual, comprising administering a subject liposomal insulin formulation.

Formulations

Liposomal formulation comprising insulin are described above. A subject liposomal insulin formulation can comprise further components, e.g., pharmaceutical excipients, which are known to those skilled in the art. Such components are disclosed in various publications, including, e.g., A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc. Additional components include, but are not limited to, an isotonicity agent, zinc, a physiologically tolerated buffer and a preservative. The physiologically tolerated buffer is preferably a phosphate buffer, such as dibasic sodium phosphate. Other physiologically tolerated buffers include TRIS, sodium acetate, or GlyGly. The selection and concentration of buffer is known in the art. Pharmaceutically acceptable preservatives include phenol, m-cresol, resorcinol, and methyl paraben.

Such liposomal formulations can be provided in any type of conventional formulation, depending on various factors, including, e.g., the route of administration. More particularly, the formulations of the present invention can be formulated into pharmaceutical compositions by combination with appropriate, pharmaceutically acceptable carriers or diluents, and may be formulated into preparations in solid, semi-solid, liquid or gaseous forms, such as tablets, capsules, powders, granules, ointments, solutions, suppositories, injections, inhalants, sprays and aerosols, as long as such formulations do not change (e.g., adversely affect) the functional integrity of the liposomes with the insulin entrapped within.

Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

For oral preparations, the formulations can be used alone or in combination with appropriate additives to make tablets, powders, granules, capsules, or buccal sprays. A variety of excipients and additives can be used, for example, conventional additives, such as lactose, propellants, mannitol, corn starch or potato starch; binders, such as crystalline cellulose, cellulose derivatives, acacia, corn starch or gelatins; disintegrators, such as corn starch, potato starch or sodium carboxymethylcellulose; lubricants, such as talc or magnesium stearate; and if desired, diluents, buffering agents, moistening agents, preservatives and flavoring agents.

The agents can be formulated into preparations for injection by suspending or emulsifying them in an aqueous or nonaqueous solvent, such as vegetable or other similar oils, synthetic aliphatic acid glycerides, esters of higher aliphatic acids or propylene glycol; and if desired, with conventional additives such as isotonic agents, suspending agents, emulsifying agents, stabilizers and preservatives.

The agents can be utilized in aerosol formulation to be administered via inhalation. The compounds of the present invention can be formulated into pressurized acceptable propellants such as dichlorodifluoromethane, propane, nitrogen and the like.

Furthermore, the agents can be made into suppositories by mixing with a variety of bases such as emulsifying bases or water-soluble bases. The compounds of the present invention can be administered rectally via a suppository. The suppository can include vehicles such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of a composition of the invention. Similarly, unit dosage forms for injection or intravenous administration may comprise a subject composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular compound employed and the effect to be achieved, and the pharmacodynamics associated with each compound in the host.

Dosages

When larger doses of insulin must be administered at a single point in time, it may be preferable to administer intermediate or long-acting insulin formulations. Such formulations typically release some insulin immediately and provide a more sustained release of the remainder of the insulin over time. It is not possible to delineate precisely the biologic responses to the various preparations because peak effects and duration vary from patient to patient and depend not only on route of administration but also on dose.

In general, dosing information for insulin via injection can be found within Harrison's—Principles of Internal Medicine (most recent edition) published by McGraw Hill Book Company, New York, incorporated herein by reference to disclose conventional information regarding dosing insulin via injection. The following paragraphs provide general guidance for insulin dosing.

The precise amount of insulin administered to a patient varies considerably depending upon the method of administration, the degree of the disease and the size of the patient. A normal-weight adult may be started on about a 15-20 units a day in that the estimated daily insulin production rate in non-diabetic subjects of normal size is approximately 25 units per day. It is preferable to administer approximately the same quantity of insulin for several days before changing the dosing regime except with hypoglycemia patients for which the dose should be immediately decreased unless a clearly evident nonrecurring cause of hypoglycemia (such as not eating, i.e., missing a typical meal) is present. In general, the changes should not be more than five to ten units per day. In some embodiments, about two-thirds of the total insulin daily dosage is administered before breakfast and the remainder administered before supper. When the total dosage reaches 50 or 60 units per day, a plurality of smaller doses are often required since peak action of insulin appears to be dose related, i.e., a low dose may exhibit maximal activity earlier and disappear sooner than a large dose. All patients are generally instructed to reduce insulin dosage by about 5 to 10 units per day when extra activity is anticipated. In a similar manner, a small amount of extra insulin may be taken before a meal that contains extra calories or food which is not generally eaten by the diabetic patient.

Obese patients are generally somewhat less sensitive to insulin and must be provided with higher doses of insulin in order to achieve the same effect as normal weight patients. Dosing characteristics based on insulin sensitivity are known to those skilled in the art and are taken into consideration with respect to the administration of insulin.

Additional information regarding dosing with insulin via injection can be found within Harrison's—Principles of Internal Medicine (most recent edition) published by McGraw Hill Book Company, New York, incorporated herein by reference to disclose conventional information regarding dosing insulin via injection.

Routes of Administration

Conventional and pharmaceutically acceptable routes of administration include intranasal, intramuscular, intratracheal, buccal, subcutaneous, intradermal, topical application, intravenous, rectal, nasal, ocular, transdermal, pulmonary, oral and other parenteral routes of administration. Routes of administration may be combined, if desired, or adjusted depending upon various factors, such as the disorder being treated, individual patient needs, etc. The subject formulation can be administered in a single dose or in multiple doses.

Combination Formulations

Patients suffering from hyperglycemia or a condition associated with hyperglycemia, e.g., diabetes mellitus, may be treated solely with an insulin formulation as indicated above. However, it is possible to treat such patients with a combination of insulin and other therapeutic agents. Accordingly, the invention provides liposomal formulations comprising insulin and one or more additional therapeutic agents. Therapeutic agents include, but are not limited to, agents that act primarily by stimulating release of insulin from the beta cells in the pancreas (e.g., a sulfonylurea drug); drugs that slow absorption and/or digestion of starches, including, but not limited to, miglitol, acarbose and other inhibitors of a-glucosidase; drugs that increase sensitivity of tissues (e.g., fat tissue, skeletal muscle tissue) to insulin, including, but not limited to, rosiglitazone, and pioglitazone. Amylin and amylin-like molecules; glucagon-like peptide-1 (GLP1) and analogs thereof; and glucagon can also be used in conjunction with insulin therapy, or therapy with insulin analogs.

Sulfonylurea drugs have the ability of increasing the number of insulin receptors in target tissues and enhance insulin-mediated glucose disposal. Some specific sulfonylurea drugs which can be used in connection with the present invention include acetohexamide administered in an amount of about 500 to 1,500 mg per day; chlorpropamide, administered in an amount of about 50 to 750 mg per day; tolazamide, administered in an amount of about 0.1 to 1 gram per day; tolbutamide, administered in an amount of about 0.5 to 3 grams per day; glipzide administered in an amount of about 2.5 to 40 mg per day and glyburide administered in an amount of about 1.25 to 20 mg per day. Other drugs include repaglinide. In patients that are producing some insulin, the sulfonylurea drugs may be sufficient to treat the symptoms.

Other patients can use a combination of the drugs while administering insulin, while still others require only the administration of insulin. The present invention is beneficial to each type of patient. Further, the present invention allows means for eliminating the need for some patients to take insulin by injection. The patients can be provided with oral doses of sulfonylureas in amounts similar to those indicated above while administering insulin via the intrapulmonary route using the formulations of the present invention. Alternatively, the patient is primarily treated by the administration of insulin via the intrapulmonary route and that treatment is supplemented by the oral administration of sulfonylureas of the type described above.

Based on the above, it will be understood by those skilled in the art that a plurality of different treatments and means of administration can be used to treat a single patient. For example, a patient can be simultaneously treated with insulin by injection, insulin via intrapulmonary administration in accordance with the present invention, and sulfonylurea drugs, which are orally administered. Benefits can be obtained by the oral administration of sulfonylurea drugs in that the insulin is naturally released by the patient in a fashion in accordance with real needs related to serum glucose levels. This natural insulin is supplemented by smaller doses provided by intrapulmonary administration in accordance with the present invention. Should such prove to be ineffective for whatever reason, such as breathing difficulties, such could be supplemented by administration via injection.

Aerosolized Delivery of Insulin Formulations

In particular embodiments, the invention provides methods of aerosolized delivery of insulin formulations to a patient in need thereof. Regardless of the active ingredient, there are several basic types of insulin formulations which can be used in connection with aerosol inhalation devices. All of the formulations include the subject liposomal formulations comprising insulin, preferably with a pharmaceutically acceptable carrier suitable for intrapulmonary administration. Any formulation which makes it possible to produce aerosolized forms of insulin which can be inhaled and delivered to a patient by the intrapulmonary route, and which do not compromise liposome integrity, can be used in connection with the present invention.

In accordance with different embodiments of the formulations, the liposomes may be a suspended in an aqueous medium for use in devices such as nebulizers. Liposomes of the invention are typically delivered as colloidal suspension in aqueous solution. In a particular embodiment, a subject liposomal formulation may be dried to obtain a powder, and the powder may be aerosolized for delivery and rehydrated in the respiratory tract. In another embodiment, the liposomes are suspended in a carrier medium, such as water, or another aqueous solution. In another embodiment, the liposomes may be suspended in a low boiling point, highly volatile propellant and optionally a pharmaceutically acceptable excipient. The liposomes can be provided in the propellant as a suspension of a dry powder or emulsions, or the liposomes can be dissolved in solution within the propellant.

Any formulation which makes it possible to produce aerosolized forms of insulin which can be inhaled and delivered to a patient via the intrapulmonary route can be used in connection with the present invention. Specific information regarding formulations which can be used in connection with aerosolized delivery devices are described within, for example, A. Gennaro (2000) “Remington: The Science and Practice of Pharmacy”, 20th edition, Lippincott, Williams, & Wilkins; Pharmaceutical Dosage Forms and Drug Delivery Systems (1999) H. C. Ansel et al., eds 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

When low boiling point propellants are used, the propellants are held within a pressurized canister of the device and maintained in a liquid state. When the valve is actuated, the propellant is released and forces the active ingredient from the canister along with the propellant. The propellant will “flash” upon exposure to the surrounding atmosphere, i.e., the propellant immediately evaporates. The flashing occurs so rapidly that it is essentially pure active ingredient that is actually delivered to the lungs of the patient.

In many embodiments, the formulations of insulin are administered into the deep part of the lung from which they cannot be removed by mucociliary clearance. Insulin is then gradually released for absorption into the systemic circulation. It is also possible to transport liposomes across into the lymphatic system and blood circulation.

Formulations of the invention can include liposomes containing insulin in combination with an amount of alveolar surfactant protein effective to enhance the transport of the liposomes across the pulmonary surface and into the circulatory system of the patient. Such liposomes and formulations containing such are disclosed within U.S. Pat. No. 5,006,343, issued Apr. 9, 1991, which is incorporated herein by reference to disclose liposomes and formulations of liposomes used in intrapulmonary delivery. The formulations and methodology disclosed in U.S. Pat. No. 5,006,343 can be adapted for the application of insulin and included within the delivery device of the present invention in order to provide for effective treatments of diabetic patients.

Regardless of the form of the drug formulation, it is preferable to create aerosolized particles of formulation having a size in the range of about 0.5 to 12 microns. By creating particles containing the liposomal formulation which particles have a relatively narrow range of size, it is possible to further increase the efficiency of the drug delivery system and improve the repeatability of the dosing. Thus, it is preferable that the particles have an aerodynamic size in the range of 0.5 to 12 microns and the mean particle size be within a narrow range so that 80% or more of the particles being delivered to a patient have a particle diameter which is within +20% of the average particle size, preferably +10% and more preferably ±5% of the average particle size. However, since the losses in the oropharyngeal cavity depend also on the inspiratory flow rate, smaller particles may be optimal for higher inspiratory flow rates whereas even bigger particles may be deposited in the deep lung when low inspiratory flow rates are used. At inspiratory flow rates between about 20 and 80 L/min, it is preferable to have particles with aerodynamic diameter in the size range between 0.5 and 4 micrometers, and preferable between 1 and 3 micrometers.

An aerosol may be created by forcing drug through pores of a membrane which pores have a size in the range of about 0.25 to 6 microns. When the pores have this size the particles which escape through the pores to create the aerosol will have a diameter in the range of 0.5 to 12 microns. Drug particles may be released with an air flow intended to keep the particles within this size range. The creation of small particles may be facilitated by the use of the vibration device which provides a vibration frequency in the range of about 800 to about 4000 kilohertz. Those skilled in the art will recognize that some adjustments can be made in the parameters such as the size of the pores from which drug is released, vibration frequency, pressure, and other parameters based on the density and viscosity of the formulation keeping in mind that the object is to provide aerosolized particles having an aerodynamic diameter in the range of about 0.5 to 12 microns.

The liposome formulation may be a low viscosity liquid formulation. The viscosity of the drug by itself or in combination with a carrier must be sufficiently low so that the formulation can be forced out of openings to form an aerosol, e.g., using 20 to 800 psi to form an aerosol preferably having an aerosolized particle size in the range of about 0.5 to 12 microns.

There are several different types of devices that use generally different mechanisms and methodologies that can be used to produce aerosols of the formulations of the invention for inhalation. The most commonly used device is a metered dose inhaler (MDI) which comprises a drug formulation container with the formulation including a low boiling point propellant. The formulation is held in the container under pressure and a metered dose of formulation is released as an aerosol when the valve on the container is opened. The low boiling point propellant quickly evaporates or “flashes” when the formulation is exposed to atmospheric pressure outside the container. The particles of formulation containing the drug without the propellant are inhaled into the patient's lungs and thereafter migrate into the patient's circulatory system. There are a number of different types of MDI devices. Devices of this type are described in U.S. Pat. Nos. 5,404,871 and 5,364,838.

Another type of aerosol delivery device forces a formulation through a porous membrane. Formulation moving through the pores breaks up to form small particles which are inhaled by the patient. Devices of this type are shown in U.S. Pat. Nos. 5,718,222, 5,554,646 and 5,522,385.

Yet another type of device is the dry powder inhaler (DPI) device. As indicated by the name such devices use formulations of dry powder in which powder is blown into an aerosolized cloud via a burst of gas. Typical DPI devices are shown in U.S. Pat. Nos. 5,458,135, 5,492,112, 5,622,166, and 5,775,320.

Other non-limiting examples of suitable devices are found in U.S. Pat. Nos. 6,158,431; 6,142,146; 6,123,068; and 6,196,218.

Further examples of devices that are suitable for delivering the formulations according to the invention include jet nebulizers, ultrasonic nebulizers, piezoelectric devices, electrospray devices, and the like.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric.

### Example 1

Acidic insulin-encapsulating multilamellar liposomes (MLV) were made from soybean phosphatidylcholine essentially by the conventional lipid-film method (Gregoriadis, Liposome Technology, volumes I-III, CRC Press, Boca Raton, Fla. (1984)). Briefly, the lipid was weighed, dissolved in chloroform, transferred to a round-bottomed flask and evaporated to dryness under low pressure using a rotary evaporator. The swelling solution, consisting of human recombinant insulin, a product lyophilized from HCl, was dissolved in water to a concentration of 40 mg/ml and added to the lipid film. The preparation was vortexed extensively for 2-5 minutes, and incubated with continuous shaking or rotating at 27° C. for two hours. At the end of the incubation period, the preparation was transferred from the round-bottomed flask to a vial appropriate for storage under regular refrigeration. The pH of the preparation was measured and found to be 2.67.

To determine the efficiency of encapsulation, aliquots of the complete preparation (i.e., containing both the encapsulated and the unencapsulated insulin) were diluted 4 fold and 60 fold, in 0.01N HCl, refrigerated for 24 hours after which they were subjected to high speed centrifugation, for 1.5 hours at 64000 g and 4° C. The centrifuge tubes, containing both the precipitated liposomes (the pellet) and the supernatant, were refrigerated again without disturbing the precipitate. Aspiration of the supernatant, containing the unencapsulated insulin, from the liposome pellet containing the encapsulated insulin, was performed 6 days later, and the pellet was resuspended in 0.01 N HCl. Insulin was assayed employing the modified Lowry method in the following systems: the complete preparation, the resuspended pellet and the supernatant. Encapsulation efficiencies of 7.8% and 19.7% were determined for 60 fold and for the 4 fold dilutions of the original preparation, respectively.

### Example 2

Acidic unilamellar liposomes (ULV) were prepared using an extrusion device (Lipex Biomembranes Inc., Vancouver, British Columbia, CA) Model T.001. An aliquot of the acidic MLV prepared in Example 1 was used as the source material and the aqueous medium was 0.01 N HCl. Extrusion was through polycarbonate membranes (a stack of 2 membranes) under nitrogen pressures of 50-100 PSI. The first extrusion was through membranes with a pore size of 1 μm, followed by seven successive extrusions through membranes with apore size of 400 nn. The liposome system underwent a 10 fold dilution in the course of the extrusion and preparation for centrifugal separation. Storage, centrifugation and determination of encapsulation efficiency were performed as described in example 1 above, yielding an encapsulation efficiency of 7.7%.

### Example 3

An aliquot of the acidic MLV prepared in Example 1 was titrated to neutrality with NaOH, and buffered by PBS to a final pH of 7.57. The aqueous media was PBS pH 7.4. Storage, centrifugation and determination of encapsulation efficiency for 30-fold and for 10-fold diluted samples (compared to the original insulin concentration of the acidic MLV) were performed as described in Example 1 above, yielding encapsulation efficiencies of 13.5% and of 31.7%, respectively.

### Example 4

Neutralized unilamellar liposomes (ULV) were prepared by extrusion as described in Example 2, using the neutralized MLV of Example 3 as source material. The liposome system underwent a 20 fold dilution (compared to the original acidic MLV) in the course of the extrusion and preparation for centrifugal separation. Storage, centrifugation and determination of encapsulation efficiency were performed as described in example 1 above, yielding an encapsulation efficiency of 14.4%.

### Example 5

Acidic insulin-encapsulating multilamellar liposomes (MLV) were prepared as in Example 1. The insulin concentration in the swelling solution was 20.25 mg/ml. The pH of the liposome preparation was 2.79. To determine the efficiency of encapsulation, aliquots from the liposome preparation were subjected to the following separation procedures: short low-speed centrifugation, gel-exclusion chromatography using micro biospin columns, and dialysis. The aqueous medium was HCl 0.01N. Encapsulation efficiency, determined as described in Example 1, was 34% by low speed centrifugation and by chromatography, and 52% by dialysis.

### Example 6

An aliquot of the preparation in Example 5 was neutralized as described in Example 2. Final pH was 7.48 and final insulin concentration was 12 mg/ml. Separation methods were low speed centrifugation and dialysis, and encapsulation efficiencies were 32% and 62%, respectively.

### Example 7

Preparation of Insulin-Encapsulating Liposomes

Steps 2, 3, 4, 7, 8 were performed each, at 5° C.

Step 1. Soybean phosphatidylcholine (SPC), at a quantity that will give a lipid concentration of 50 mg/ml for the final liposome product, was weighed and dissolved in chloroform. The solution was transferred to a round-bottomed flask and evaporated to dryness under low pressure in a rotary evaporator, at 38° C.

Step 2. A GlyGly solution was prepared at a concentration that would reach the value of 2.64 mg/ml in the final liposomal product.

Step 3. Preparation of the insulin solution: The desired amount of insulin (supplied by Novo Nordisk, regular human recombinant Zn-insulin), to make a final concentration of 40 mg/ml, was weighed and dissolved in the GlyGly solution, to a volume that was 50-70% that of the final volume set for the batch.

Step 4. The insulin solution was titrated with NaOH to pH 9.0.

Step 5: This insulin solution, denoted the “swelling solution” was incubated at 4° C. for 20 minutes to ensure complete dissolution.

Step 6. The swelling solution was added to the thin lipid layer in the round-bottomed flask and subjected to intensive vortexing for several minutes, followed by incubation in a rotating shaker, in a 37° C. temperature room, for two hours.

Step 7. Upon termination of incubation, the pH was adjusted to range of 7.4-7.6 by titration with HCl.

Step 8. NaCl, at a quantity to make its concentration in the final liposome product 150 mM was weighed and added to the neutralized liposome preparation of step 7.

Step 9. The final volume and pH were recorded, and the liposome suspension was refrigerated until further use.

Determination of Encapsulation Efficiency

Terminology

Complete preparation: the liposome preparation as it comes off production, containing both encapsulated and unencapsulated insulin.

Encapsulated insulin: The liposomal pellets obtained from separation by centrifugation, that contain only encapsulated insulin, re-suspended in an insulin-free buffered aqueous solution.

Unencapsulated insulin: the supernatant obtained from the separation process

Definition

The efficiency of encapsulation is defined as the ratio of concentrations of encapsulated insulin, to that of insulin in the complete preparation.

Performing the Centrifugation

(a) small-size samples: Aliquots of 200 μl of the liposome preparation were subjected to centrifugation in a table top mini ultracentrifuge operated at 4° C., for 30 minutes, at a g force of 130000. This g force is substantially higher than is needed to spin down liposomes of the MLV type.

(b) Medium-sized samples: Aliquots of 600 μl, were subjected to centrifugation in a table top mini high-speed centrifuge operated at 4° C. for 60 minutes, at a g force of 20000.

The supernatant was carefully aspirated and saved and the pellet was re-suspended to original aliquot volume in an appropriate insulin-free aqueous medium buffered to the same pH as the complete (neutralized) preparation.

Insulin Assays

Two assays were used to determine the insulin concentrations in: buffered aqueous solutions; the complete liposome preparation; the encapsulated insulin fraction (i.e., the re-suspended liposomal pellets); the unencapsulated insulin fraction (i.e., the supernatant): (1) Modified Lowry and (2) insulin absorbance at 280 nm (“UV”). The assays were performed in 96 well plates using, for the insulin UV absorbance, plates specially suited for that range. The plate reader was Thermomax Microplate Autoreader from Molecular Devices.

Calculation of Encapsulation Efficiency

The concentration of encapsulated insulin, and the concentration of the unencapsulated insulin were calculated from the assays of insulin in the liposomal pellets and in the supernatant, respectively. The total insulin concentration of the complete preparation, to be referred to as “total insulin” was calculated by: (i) insulin assay of samples from the complete preparation and (ii) summing up the concentrations of encapsulated and un-encapsulated insulin. The efficiency of encapsulated was then calculated from the concentration ratio of encapsulated to total insulin, taking the latter once from (i) above and once from (ii) above.

Results

Insulin concentration in the final preparation was 37.2(±1.0) mg/ml. Encapsulation efficiency was 15.2(±1.1) %.

### Example 8

Insulin-encapsulating liposomes of the MLV type were prepared, and encapsulation efficiency determined, as described in example 7 above, except the following change: The incubation of the insulin solution at pH=9, detailed in step 5 of example 7, was increased to 30 minutes. Insulin concentration in the final preparation was 37.3(±0.8) mg/ml. Encapsulation efficiency was 16.3(±1.9) %.

### Example 9

Insulin-encapsulating liposomes of the MLV type were prepared, and encapsulation efficiency determined, as described in Example 7 above, except the following change: The incubation of the insulin solution at pH=9, detailed in step 5 of example 7, was increased to 2 hours. Insulin concentration in the final preparation was 38.0(±0.8) mg/ml. Encapsulation efficiency was 24.5(±1.3) %.

### Example 10

Insulin-encapsulating liposomes of the MLV type were prepared, and encapsulation efficiency determined, as described in Example 7 above, except the following change: The incubation of the insulin solution at pH=9, detailed in step 5 of example 7, was increased to 18 hours. Insulin concentration in the final preparation was 35.6(±1.6) mg/ml. Encapsulation efficiency was 50.3(±5.0) %.

### Example 11

Liposome Preparation

Insulin-encapsulated liposomes of the MLV type were prepared as described in Example 7 above, except the following changes: (1) Step 8, the addition of NaCl was moved forward to the end of step 4, before 5 and (2) The incubation period in step 5 was 18 hours.

Methodologies

Efficiency of encapsulation was determined by two separate and independent experimental approaches: (1) as detailed in Example 7, through separation of the complete preparation by centrifugation. The approach will be denoted “the thermodynamic” and (2) by an approach denoted “the kinetic”, which is described below.

The Kinetic Approach: Insulin Efflux from Liposomes

A suspension of liposomes (0.5-1.0 ml) was placed in a dialysis sac (MW cutoff 12000-14000 Da), and the sac was immersed in a constantly-stirred receiver vessel which contained insulin-free GlyGly/saline buffer at pH=7.6. at volumes 10 to 16 fold that of the sac. At designated periods, the dialysis sac was transferred from one receiver vessel to another, containing fresh (i.e., drug-free) buffer. Insulin concentration was assayed in each dialysate and in the sac (at the beginning and end of each experiment). For cases in which the insulin concentration of the dialysate was below or at the lower edge of detection limits, the dialysates were concentrated by lyophilization as follows: 1 ml Aliquots were lyophilized to dry powder and were re-dissolved in 100-200 μl of water. Buffer aliquots as well as insulin calibration standards were subjected to the same process.

In order to obtain a quantitative evaluation of drug release, experimental data were analyzed according to a previously-derived multi-pool kinetic model (Margalit, R., Alon, R., Linenberg, M., Rubin, I., Roseman, T. J. and Wood R. W. (1991) J. Control. Rel., 17: 285-296), in which drug efflux from the sac into the reservoir is the product of a series of independent drug pools, one corresponding to free (i.e., unencapsulated), and all others to liposome-associated drug. The overall drug released at time=t, f(t), should correspond to the following equation:

$$f ( t ) = {\sum }_{j = 1}^{n} {f}_{j} ( 1 - {exp}^{- {k}_{j} t} ) \qquad ( 1 )$$

where fⱼ is the fraction of the total drug in the system occupying the j'th pool at time=0, and kⱼ is the rate constant for drug diffusion from the j'th pool.

When the samples for dialysis were the complete preparation, the value of fⱼ, for the encapsulated insulin, is also the value of the encapsulation efficiency.

Results

The original preparation was at the lipid concentration of 50 mg/ml and the swelling solution contained 40 mg/ml insulin, as in Examples 7-10 above. Insulin concentration in the final preparation was 37.6(±1.7) mg/ml. Encapsulation efficiency was 93.3(±3.6)% and 87.3(±0.3)%, as determined by the thermodynamic and kinetic approaches, respectively. These results are highly satisfactory with respect to the encapsulation level, and also show good agreement between the two approaches.

The original preparation was diluted 1/20 into insulin-free GlyGly/saline buffer at pH=7.6, yielding a preparation with a liposome concentration of 2.5 mg lipid/ml. Insulin concentration in this (diluted) final preparation was 2.1 (±0.2) mg/ml, in good agreement with the theoretical value of 2.0 mg/ml. Encapsulation efficiency of 84.6(±0.5)% was determined by the kinetic approach. This indicates that the systems perform as sustained release insulin depots, and that there is no risk of immediate loss of encapsulated insulin upon dilution of the liposomal preparation. These desired traits will be further substantiated in examples reporting results of insulin efflux kinetics.

### Example 12

Liposome Preparation

Insulin-encapsulating Unilamellar Liposomes (ULV) were prepared by a two-step process. First, MLV were prepared as described in example 1. Liposome (MLV) concentration was 50 mg/ml, and insulin concentration of the swelling solution was 40 mg/ml. The complete MLV preparation was the “raw material” for the second step, which consisted of extruding the liposomes through a LIPEX extrusion device, as described in example 2, save the final 7 cycles were through membranes with a pore size of 450 μM. Methodologies, definitions and calculations were as described in Examples 7 and 11.

Results

The final liposome preparation was 21 mg lipid/ml. Based on this the theoretical expectation for insulin concentration of the final preparation was 16.8 mg/ml and the experimentally-determined value was 17.5(±0.6) mg/ml, in quite good agreement. An encapsulation efficiency of 86.7(±0.2)%, was determined by the kinetic approach.

### Example 13

Insulin-encapsulating MLV were prepared, and encapsulation efficiency determined, as described in example 11 above, except the lipid concentration was reduced to 25 mg/ml. Insulin concentration in the final preparation was 31.9(±1.7) mg/ml. An encapsulation efficiency of 21.5(±4.5) %, was determined by the thermodynamic approach.

### Example 14

Insulin-encapsulating MLV were prepared, and encapsulation efficiency determined, as described in Example 11 above, except the insulin concentration of the swelling solution was raised to 60 mg/ml. Insulin concentration in the final preparation was 55.1(±4.4) mg/ml. An encapsulation efficiency of 36.0(±2.9) %, was determined by the thermodynamic approach.

### Example 15

Insulin-encapsulating MLV were prepared, and encapsulation efficiency determined, as described in Example 11 above, except the lipid concentration was raised to 100 mg/ml. Insulin concentration in the final preparation was 38.4(±2.6) mg/ml. Encapsulation efficiency was 82.8(±4.7) % and 92.5(±1.0)%, as determined by the thermodynamic and kinetic approaches, respectively. As in Example 11, these results are highly satisfactory with respect to the encapsulation level, and also show good agreement between the two approaches.

The original preparation was diluted 1/20 into insulin-free GlyGly/saline buffer at pH=7.6, yielding a preparation with a liposome concentration of 5.0 mg lipid/ml. Insulin concentration in this (diluted) final preparation was 2.1 (±0.1) mg/ml, in good agreement with the theoretical value of 2.0 mg/ml. Encapsulation efficiency of 88.9(±0.3)% was determined by the kinetic approach. As in Example 11, this indicates that the systems perform as sustained release insulin depots, and that there is no risk of immediate loss of encapsulated insulin upon dilution of the liposomal preparation.

### Example 16

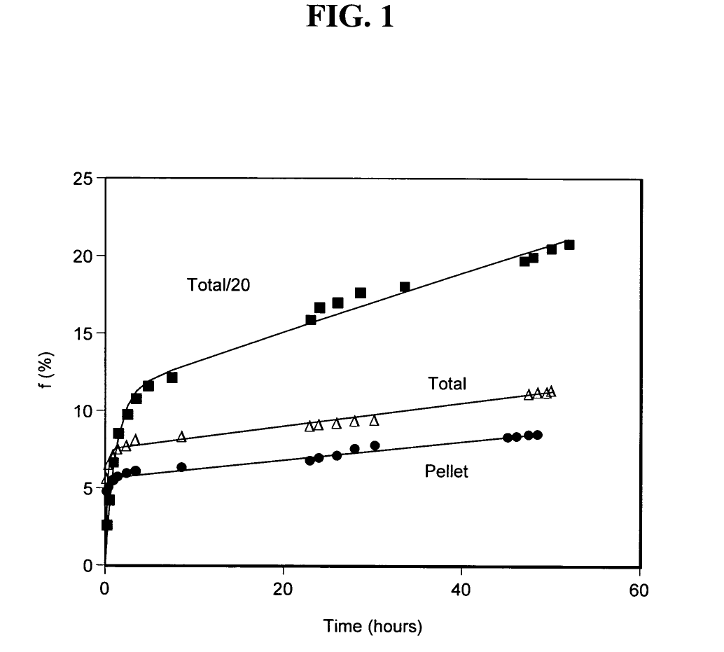

Insulin efflux kinetics, performed according to the methodology detailed in Example 11, were studied for the following types of samples obtained, from the liposomes of Example 15: (1) the complete original preparation (2) the “encapsulated insulin” from the original preparation (see terminology in example 7) and (3) the complete diluted preparation. The results for all three systems are shown in FIG. 1. The experimental data (represented by the symbols in FIG. 1) were processed according to equation (1) presented in Example 11 above, and were found to fit the case of two independent drug pools:

f(t)=f₁(1−exp^{-k}_{₁}^{t})+f₂(1−exp^{-k}_{₂}^{t}) (2)

Where the indices 1 and 2 are for the faster and slower efflux rates, and are assigned to unencapsulated insulin and encapsulated insulin, respectively. The solid curves in FIG. 1, are the theoretical expectations according to equation (2). Both the experimental and theoretical data, that are in good agreement over the entire 2-day time span of the experiment, make it clear that the liposomal-insulin formulations behave as sustained-release insulin depots.

### Example 17

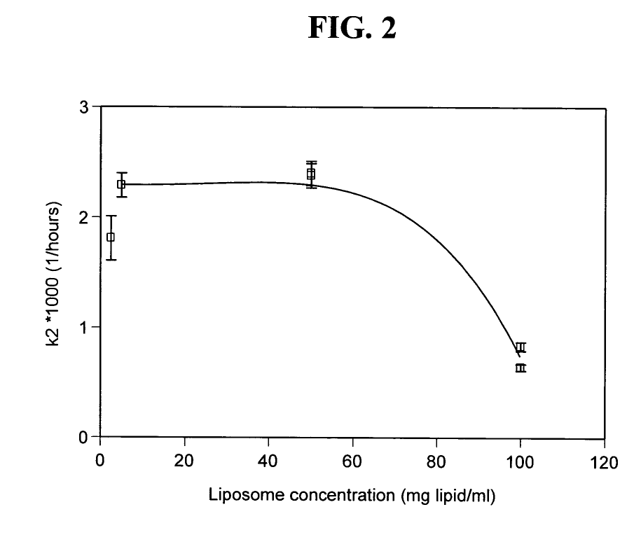

Kinetic experiments of the type described in Example 16 were conducted for a series of insulin-encapsulating MLV, prepared essentially according to Example 11, but with different liposome concentrations spanning the range of 2.5-100 mg lipid/ml. The data were processed as in example 16 above, and the rate constants for the efflux of encapsulated insulin (k₂) are plotted in FIG. 2, as function of liposome concentration. The decrease in k₂ with the increase in liposome concentration fits the previously-derived theoretical expectations that were furthermore supported experimentally for a host of encapsulated matters that span a molecular weight range from 100-6000 Da (Margalit, R., Alon, R., Linenberg, M., Rubin, I., Roseman, T. J. and Wood R. W. (1991) J. Control. Rel., 17: 285-296; Margalit, R. and Yerushalmi, N. (1996). In: “Microencapsulation Methods and Industrial Applications.” Benita S., ed., Chapter 10, 59-295. Marcel Dekker Inc).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method for preparing an insulin formulation, the method comprising the steps of:
(a) preparing a solution comprising insulin, wherein the solution has a non-neutral pH;
(b) encapsulating the solution in liposomes at a non-neutral pH; and
(c) neutralizing the liposomes of step (b) to pH of 7.2 to 7.6.

2. The method of claim 1, wherein the solution has a pH between about 2.3 and 3.0 before it is neutralized, and wherein the encapsulation takes place in an acidic environment.

3. The method of claim 1, wherein the solution has a pH between about 7.8 and 9.5 before it is neutralized, and wherein the encapsulation takes place in a basic environment.

4. The method of claim 1, wherein the solution of step (a) comprises 20 to 80 mg/ml insulin.

5. The method of claim 1, wherein the liposomes are between about 0.2 and 3 microns in diameter.

6. The method of claim 1, wherein the liposomes comprise a lipid selected from the group consisting of: phosphatidylcholine, phosphatidyl ethanolamine, cholesterol, phosophatidyl serine, phosphatidyl glycerol and phosphatidyl inositol.

7. The method of claim 1, wherein the liposomes are unilamellar.

8. The method of claim 1, wherein the liposomes are multilamellar.

9. A composition comprising monomeric insulin encapsulated in liposomes, wherein the monomeric insulin has a molecular weight of about 6,000 Da.

10. The composition of claim 9, wherein the insulin is an insulin analog.

11. The composition of claim 9, wherein the insulin is selected from the group consisting of a superactive insulin analog, a monomeric insulin analog, and a hepatospecific insulin analog.

12. The composition of claim 11, wherein the insulin is insulin lispro.

13. The composition of claim 1, wherein the solution further comprises a second therapeutic agent.

14. A method for reducing a blood glucose level in a patient, the method comprising administering a formulation comprising monomeric insulin encapsulated in liposomes to the patient.

15. The method of claim 14, wherein the monomeric insulin has a molecular weight of about 6,000 Da.

16. The method of claim 14, wherein the administering is carried out by:
creating an aerosol of the formulation; and
inhaling the aerosol.

17. The method of claim 14, wherein the administering is carried out by:
creating an injectable solution of the formulation; and
injecting the solution.

18. A method for treating a condition related to hyperglycemia in an individual, the method comprising administering a formulation comprising monomeric insulin encapsulated in liposomes to the individual.

19. A method of delivering insulin to a lung of an individual, comprising:
preparing an insulin formulation by the method of claim 1;
aerosolizing the insulin formulation to provide aerosol particles having an aerodynamic diameter of about 1 to about 5 microns; and
inhaling the aerosol into the lung of the individual.
